(19) United States
(12) Patent Application Publication
(10) Pub. No.: US 2019/0321120 A1
(43) Pub. Date: Oct. 24, 2019

(54) Film Sheath with Built-In Flange that Allows for Easy and Secure Attachment or Bonding

(71) Applicants: Stephen E. BONDURA, GREENVILLE, SC (US); AVENT, INC., Alpharetta, GA (US)

(72) Inventors: Ross A. Hutchison, Canton, GA (US); Robert Dean Peterson, Jr., Roswell, GA (US); Ajay Y. Houde, Alpharetta, GA (US)

(21) Appl. No.: 16/312,725

(22) PCT Filed: Jun. 28, 2016
(86) PCT No.: PCT/US2016/039765

§ 371 Date: Dec. 21, 2018

(51) Int. Cl. A61B 46/17

(52) U.S. Cl. CPC A61B 46/17; A61B 90/40

(57) ABSTRACT

A probe cover is provided. The probe cover is formed from a film that defines a tubular sheath having a distal end and a proximal end defining a channel therebetween, where the distal end is closed and the proximal end defines an opening having a periphery; and a flange that extends from the periphery of the opening at the proximal end of the tubular sheath. The flange facilitates the secure attachment of the probe cover to a surgical drape, such as a surgical drape used in the operating room as an anesthesia screen or as a screen to separate the surgical sterile field from the non-sterile field. A method of using the probe cover in conjunction with a probe during a surgical procedure is also provided, where the method includes attaching the probe cover to a surgical drape. A surgical drape that includes the probe cover is further provided.

### FIELD OF THE INVENTION

The present invention relates to a probe cover for use in a surgical environment, where the probe cover is configured for attachment to a surgical drape to create a channel through which a non-sterile medical instrument can be introduced from a non-sterile field into a sterile surgical field.

### BACKGROUND OF THE INVENTION

In the operating room, surgeons, anesthesiologists, nurses, and other healthcare professionals may often be required to use medical instruments such as ultrasound machines, electrocardiogram machines, internal cardiac defibrillator wands, etc. However, these medical instruments are not sterile, and introducing probes, wands, or other components of the medical instruments into the sterile surgical field in order to assess a patient's condition or provide treatment to the patient can put the patient at risk for an infection or other complications. Thus, in order to introduce the aforementioned probes, wands, etc. into the sterile field, probe covers have been developed from film-like materials, where the probe covers have a closed end that receives the probe and an open end that receives the probe and a cord that connects the probe to the medical instrument with which it is associated. The probe cover, the probe, and the portion of the cord or other material attached to the probe are thus surrounded by the cover and can then been introduced to the sterile surgical field, while the equipment itself and the remaining portion of the cord that is not surrounded by the cover can remain in the non-sterile field. Typically, the assembly of a probe surrounded by a sterile probe cover is introduced into the sterile field of the surgical site by either draping the assembly over an anesthesia screen formed from a surgical drape or by draping it over the side of the surgical drape. However, this practice leads to a high risk of contamination of the sterile field, thus increasing the potential occurrence of surgical site infections.

As such, U.S. Patent Application Publication No. 2013/0247921 describes the use of a surgical drape that can be used as an anesthesia screen, where the surgical drape is used in conjunction with a portal that is used to create a channel through the surgical drape. The portal includes an applicator frame and a separate probe cover. The applicator frame is connected or attached to the surgical drape, and then the separate probe cover is attached to the applicator frame. However, attaching the probe cover to the applicator frame that has been connected or attached to the surgical drape is cumbersome and time consuming, and an insecure attachment of the probe cover can result if not performed carefully.

Thus, a need exists for an improved probe cover that can be securely and easily attached to a surgical drape to cover probes or other components of medical instruments and equipment that must pass from a non-sterile field into a sterile surgical field.

### SUMMARY OF THE INVENTION

In accordance with one embodiment of the present invention, a probe cover is provided. The probe cover is formed from a film, wherein the film defines a tubular sheath having a distal end and a proximal end defining a channel therebetween, wherein the distal end is closed and the proximal end defines an opening having a periphery; and a flange extending from the periphery of the opening at the proximal end of the tubular sheath.

In one particular embodiment, the flange can be adapted to attach to a planar or semi-planar surface.

In another embodiment, the tubular sheath and the flange can be flexible.

In still another embodiment, an adhesive can be disposed on a non-sterile field facing surface of the flange or a sterile surgical field facing surface of the flange. Further, a release liner can be disposed on the adhesive. In addition, the adhesive can be a hot melt adhesive coating, an acrylic adhesive coating, a tape, or a combination thereof.

In yet another embodiment, the probe cover can be configured for direct attachment to a surgical drape.

In an additional embodiment, the film can be seamless.

In one more embodiment, the film can include latex, polyurethane, polyisoprene, polycarbonate, polypropylene, polyethylene, or a combination thereof.

In another embodiment, the flange can extend from the periphery of the opening at the proximal end of the tubular sheath to opposing first and second edges and opposing third and fourth edges such that the flange has a square or rectangular shape.

In still another embodiment, the flange can have a surface area that is sufficiently large to cover an opening in a surgical drape to which the flange is attached.

In yet another embodiment, the channel can receive a probe connected to a non-sterile medical instrument. Further, the probe can be an ultrasound probe, an echocardiography probe, or an internal cardiac defibrillator wand.

In accordance with another embodiment of the present invention, a method for using a probe in a sterile surgical field, wherein the probe is a component of a non-sterile medical instrument located in a non-sterile field, is provided. The method includes positioning a probe cover adjacent a sterile surgical field side or a non-sterile field side of a surgical drape material, wherein the probe cover comprises a film, wherein the film defines a tubular sheath having a distal end and a proximal end defining a channel therebetween, wherein the distal end is closed and the proximal end defines an opening; and a flange extending from the periphery of the opening at the proximal end of the tubular sheath; attaching a non-sterile field facing surface of the flange to a sterile surgical facing surface of the surgical drape material or attaching a sterile field facing surface of the flange to a non-sterile field facing surface of the surgical drape material; and inserting the probe into the opening at the proximal end of the tubular sheath and into the channel from the non-sterile field, wherein the probe is connected to the non-sterile medical instrument via a cord such that the probe is encased by the tubular sheath.

In one particular embodiment, an adhesive can be disposed on the non-sterile field facing surface of the flange or on the sterile surgical field facing surface of the flange. Further, a release liner can be disposed on the adhesive, wherein the release liner is removed prior to attaching the non-sterile field facing surface of the flange to the sterile surgical field facing surface of the surgical drape material or prior to attaching the sterile-field facing surface of the flange to the non-sterile field facing surface of the surgical drape material.

In another embodiment, the method further includes the step of cutting an opening in the surgical drape material at a location where the flange is attached such that the opening at the proximal end of the tubular sheath lines up with the opening cut in the surgical drape material. Further, the flange can have a surface area that is sufficiently large to cover the opening in the surgical drape material.

In still another embodiment, the method further includes the step of advancing the probe contained within the channel and encased by the tubular sheath into the sterile surgical field for use on a patient.

In yet another embodiment, the film can be seamless.

In an additional embodiment, the tubular sheath and flange can be flexible such that the tubular sheath is movable in multiple planes within the sterile surgical field and such that the flange is attachable to a planar or semi-planar surface.

In one more embodiment, the probe can be an ultrasound probe, an echocardiography probe, or an internal cardiac defibrillator wand.

In accordance with one more embodiment of the present invention, a surgical drape for separating a sterile surgical field from a non-sterile field is provided. The surgical drape includes a surgical drape material having an opening formed therein and a probe cover formed from a film, wherein the film defines a tubular sheath having a distal end and a proximal end defining a channel therebetween, wherein the distal end is closed and the proximal end defines an opening having a periphery; and a flange extending from the periphery of the opening at the proximal end of the tubular sheath, wherein the probe cover opening is aligned with the surgical drape material opening, wherein the probe cover is attached to the surgical drape material via the flange.

These and other features, aspects and advantages of the present invention will become better understood with reference to the following description and appended claims. The accompanying drawings, which are incorporated in and constitute a part of this specification, illustrate embodiments of the invention and, together with the description, serve to explain the principles of the invention.

### BRIEF DESCRIPTION OF THE FIGURES

A full and enabling disclosure of the present invention to one skilled in the art, including the best mode thereof, is set forth more particularly in the remainder of the specification, including reference to the accompanying figures, in which:

FIG. 1 illustrates a side view of one embodiment of the probe cover of the present invention;

FIG. 2 illustrates a perspective view of the probe cover of the present invention;

FIG. 3 illustrates another side view of one embodiment of the probe cover of the present invention;

FIG. 4 illustrates a side view of the probe cover over the present invention when the probe cover is attached to a surgical drape material on a sterile surgical field facing surface; and

FIG. 5 illustrates a side view of the probe cover over the present invention when the probe cover is attached to a surgical drape material on a non-sterile facing surface.

Repeat use of reference characters in the present specification and drawings is intended to represent the same or analogous features or elements of the present invention.

### DETAILED DESCRIPTION OF REPRESENTATIVE EMBODIMENTS

Reference now will be made in detail to various embodiments of the invention, one or more examples of which are set forth below. Each example is provided by way of explanation of the invention, not limitation of the invention. In fact, it will be apparent to those skilled in the art that various modifications and variations may be made in the present invention without departing from the scope or spirit of the invention. For instance, features illustrated or described as part of one embodiment, may be used on another embodiment to yield a still further embodiment. Thus, it is intended that the present invention covers such modifications and variations as come within the scope of the appended claims and their equivalents.

Generally speaking, the present invention is directed to a probe cover that is formed from a film that defines a tubular sheath having a distal end and a proximal end defining a channel therebetween, where the distal end is closed and the proximal end defines an opening having a periphery, and a flange that extends from the periphery of the opening at the proximal end of the tubular sheath. The flange facilitates the secure attachment of the probe cover to a surgical drape, such as a surgical drape used in the operating room as an anesthesia screen or as a screen to separate the surgical sterile field from the non-sterile field. A method of using the probe cover in conjunction with a probe during a surgical procedure is also provided, where the method includes attaching the probe cover to a surgical drape. A surgical drape that includes the probe cover is further provided.

Specifically, the flange component of the probe cover is continuous with the tubular sheath component, as both components are formed from the same film. The flange allows for easy, direct attachment of the probe cover to a planar or semi-planar surface such as a surgical drape used as an anesthesia screen or a screen to separate the sterile surgical field from the non-sterile field that includes non-sterile equipment (e.g., medical instruments such as an ultrasound machine, electrocardiography machine, etc.) and other items. In other words, the flange can be described as extra film material present at the opening at the proximal end of the tubular sheath that extends from the periphery of the opening at a generally perpendicular angle to the tubular sheath, where the flange allows the probe cover to be directly attached to the surgical drape. Once the probe cover is attached to the surgical drape, an ultrasound probe, an echocardiography probe, an internal cardiac defibrillator wand, etc. can be passed from the non-sterile field on one side of the surgical drape, through an opening in the surgical drape, and then through the opening in the probe cover, so that the probe, wand, etc. is surrounded by the probe cover when it enters the sterile surgical field on the other side of the surgical drape. The probe cover allows for seamless introduction of the probe or wand into the sterile surgical field and reduces the risk on contamination that arises by draping the probe cover over the surgical drape, which, in turn, reduces the risk of a patient having a surgical site infection.

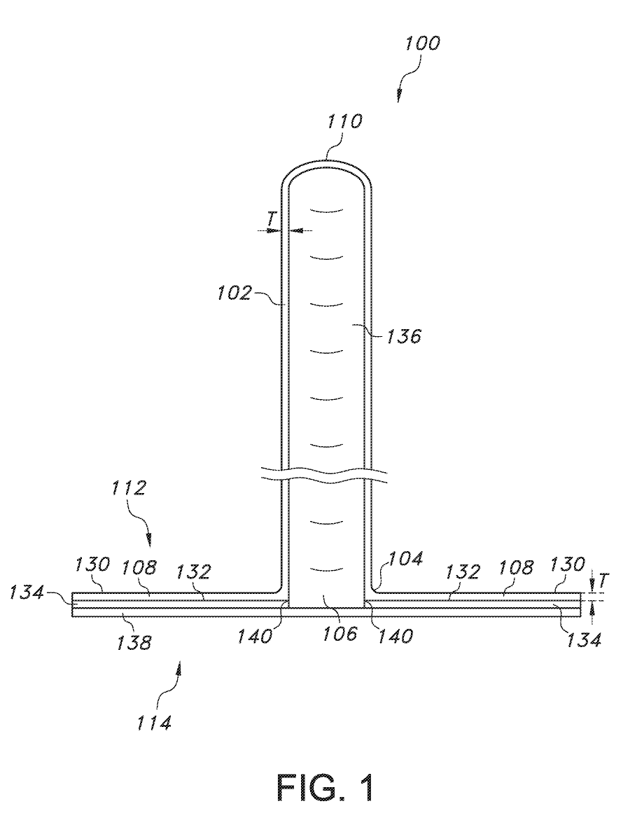

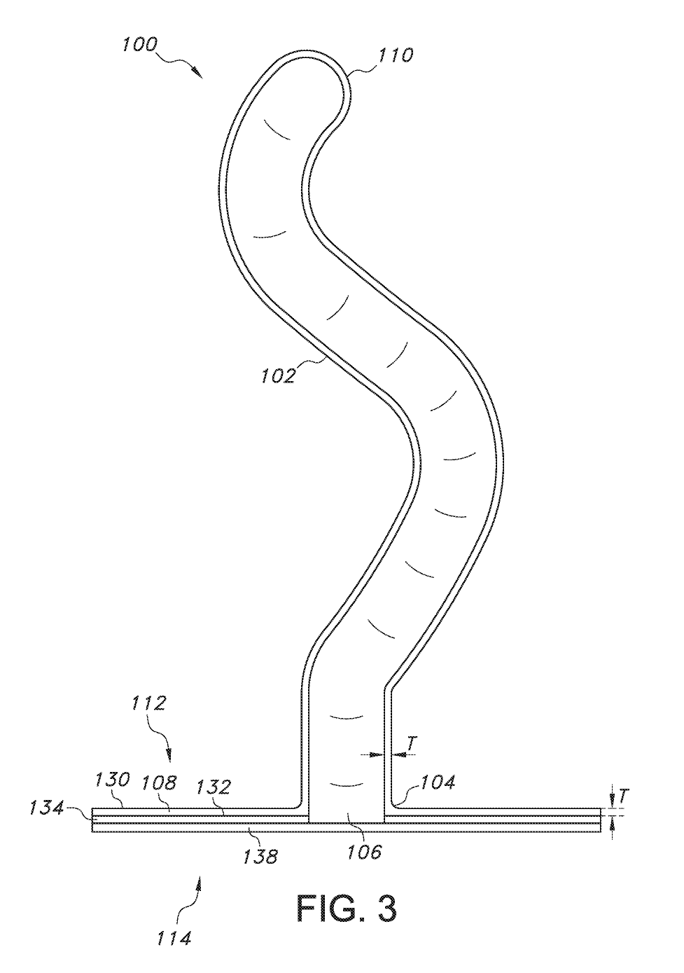

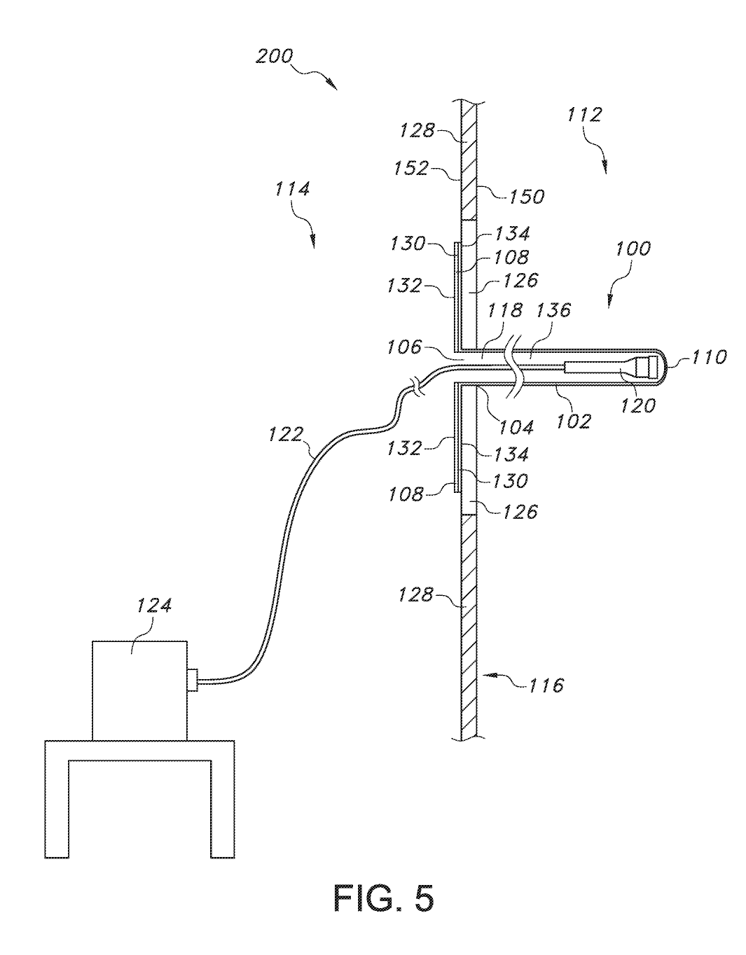

Turning now to FIGS. 1 and 3, one embodiment of a probe cover 100 is shown. The probe cover 100 includes a tubular sheath 102 having a closed distal end 110 and a proximal end 104 that defines an opening 106. Further, the distal end 110 and the proximal end 104 define a channel 136 into which a probe that is connected to a non-sterile medical instrument located in a non-sterile field 114 can be inserted, where the distal end 110 extends into a sterile surgical field 112. The probe cover 100 also includes a flange 108 that extends from the periphery 140 of the opening 106 at the proximal end 104 of the tubular sheath 102. The flange 108 includes a sterile surgical field facing surface 130 and a non-sterile field facing surface 132. In some embodiments, an adhesive 134 can be coated onto or otherwise disposed on or attached to the non-sterile field facing surface 132 of the flange 108, where the adhesive 134 can be a hot melt adhesive, an acrylic adhesive, a tape, or a combination thereof. Further, a release liner 138 can be disposed on the adhesive 134 to protect the adhesive 134 until the probe cover 100 is attached to a surface, such as a surgical drape material 116. In other embodiments, it is to be understood that an adhesive 134 can be coated onto or otherwise disposed on or attached to the sterile surgical field facing surface 130 of the flange 108, where the adhesive 134 can be a hot melt adhesive, an acrylic adhesive, a tape, or a combination thereof. Further, a release liner 138 can be disposed on the adhesive 134 to protect the adhesive 134 until the probe cover 100 is attached to a surface, such as a surgical drape material 116 (see FIG. 5).

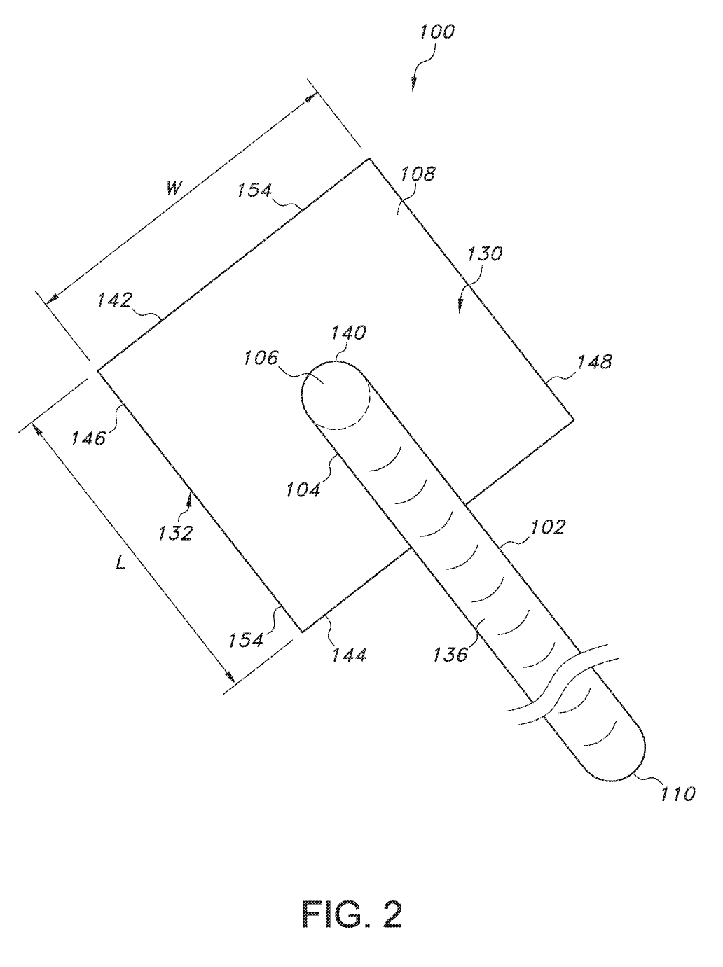

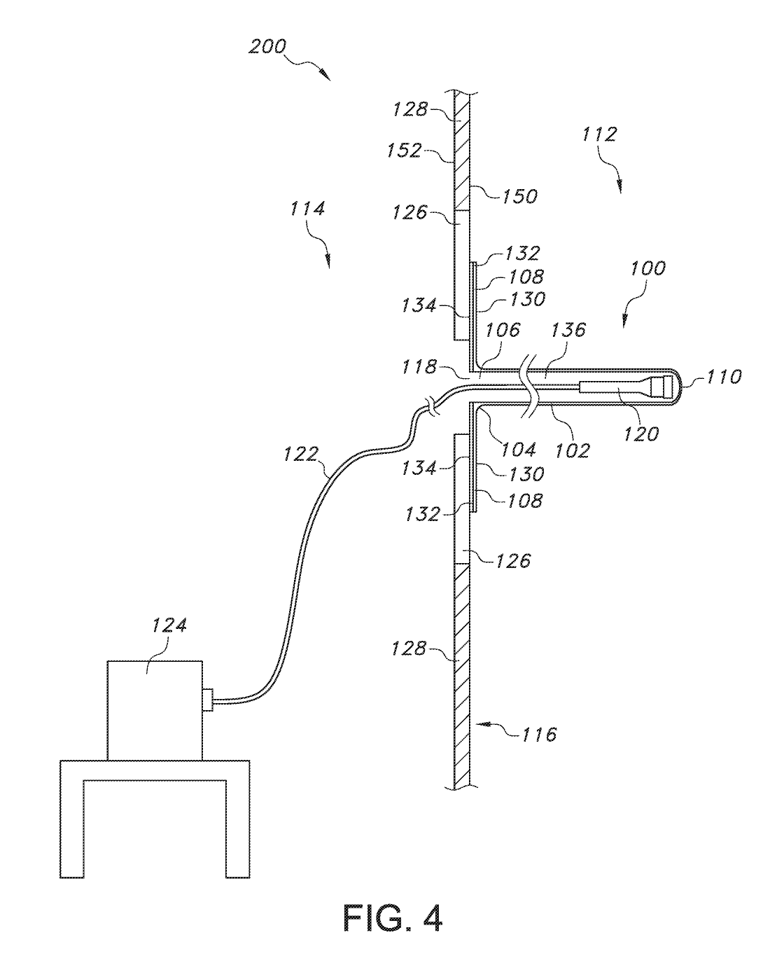

Referring to FIG. 2, in some embodiments, the flange 108 can extend from the periphery 140 of the opening 106 at the proximal end 104 of the tubular sheath 102 to opposing first edge 142 and second edge 144 as well as opposing third edge 146 and fourth edge 148 such that the flange 108 has a square or rectangular shape. However, it is also to be understood that the flange can have any other suitable shape, such as circular, elliptical, oval, triangular, etc. The flange 108 can be adapted to directly attach to a planar or semi-planar surface such as a surgical drape in that it is generally planar but sufficiently flexible to conform and attach to a semi-planar surface if needed. As shown in FIG. 3, the tubular sheath 102 and flange 108 can be sufficiently flexible such that the tubular sheath 102 is movable in multiple planes within the sterile surgical field 112 and such that the flange 108 can be attached to a planar or semi-planar surface. Moreover, the flange 108 has a surface area that is sufficiently large to cover an opening 118 in a surgical drape to which the flange 108 can be directly attached (see FIG. 4). In this regard, the outer periphery 154 of the flange 108 can have a length L ranging from about 60 millimeters to about 250 millimeters, such as from about 75 millimeters to about 150 millimeters, such as from about 90 millimeters to about 130 millimeters. In addition, the outer periphery of the flange 108 can have a width W ranging from about 60 millimeters to about 250 millimeters, such as from about 75 millimeters to about 150 millimeters, such as from about 90 millimeters to about 130 millimeters. Meanwhile, the opening 106 can have a diameter ranging from about 40 millimeters to about 150 millimeters, such as from about 50 millimeters to about 130 millimeters, such as from about 60 millimeters to about 100 millimeters. Further, the ratio of the length L or width W of the flange 108 to the diameter of the opening 106 can range from about 1.1 to about 6, such as from about 1.2 to about 4, such as from about 1.3 to about 2.

As shown in FIGS. 1 and 3 and as mentioned above, the probe cover 100 can be flexible or somewhat rigid, and can be formed from any suitable film material. For instance, the probe cover, including the tubular sheath 102 and the flange 108, can be formed from any suitable material such as latex, polyurethane, polyisoprene, polycarbonate, polypropylene, polyethylene, or a combination thereof. Further, the film material from which the probe cover 100 is formed can be seamless in that there is no discontinuity between the proximal end 104 of the sheath 102 and the flange 106. In addition, the film can have a thickness T that ranges from about 3 millimeters to about 20 millimeters, such as from about 5 millimeters to about 15 millimeters, such as from about 6 millimeters to about 12 millimeters.

Turning now to FIG. 4, in another particular embodiment, the probe cover 100 of FIGS. 1-3 can be a component of a surgical drape 200 along with a surgical drape material 116, which can include any material used to form a surgical drape (nonwoven materials such as meltblown webs, spunbond webs, spunbond-meltblown-spunbond laminates, etc.). Meanwhile, FIG. 4 illustrates how the probe cover 100 can receive a probe 120 that is connected to a non-sterile medical instrument 124 present in the non-sterile field 114 via a cord 122 so that the probe 120 can be used in the sterile surgical field 112 on the opposite side of a surgical drape 200. Specifically, the surgical drape 200 includes a surgical drape material 116, which can be formed of any nonwoven material or other material known in the art that is used as a surgical drape in the operating room. The surgical drape material 116 has a sterile surgical field facing surface 150 and a non-sterile surgical field facing surface 152. In addition, although not required, the surgical drape material 116 can include a translucent section 126 and an opaque section 128, where the translucent section 126 allows an anesthesiologist or other healthcare professional to view the patient and/or procedures being performed in the sterile surgical field 112. An opening 118 can also be formed in the surgical drape material 116, such as, for example, in the translucent section 126, so that the probe 120 can be passed through the drape 200 from the non-sterile field 114 into the sterile surgical field 112. As shown, in one particular embodiment, the probe cover 100 is directly attached to the surgical drape material 116 on its sterile surgical field facing surface 150 via an adhesive 134 present on the non-sterile field facing surface 132 of the flange 108 so that the opening 106 at the proximal end 104 of the probe cover 100 is generally aligned with the opening 118 in the surgical drape material 116. However, referring to FIG. 5, it is also to be understood that the probe cover 100 can be directly attached to the surgical drape material 116 on its non-sterile field facing surface 152 via an adhesive 134 present on the sterile-surgical field facing surface 130 of the flange 108 so that the opening 106 at the proximal end 104 of the probe cover 100 is generally aligned with the opening 118 in the surgical drape material 116. Although the opening 118 in the surgical drape material 116 can be pre-formed, in some embodiments, the opening 118 can be cut into the surgical drape material 116 at the time of use or assembly of the surgical drape material/probe cover combination in the operating room. In any event, after the probe cover 100 has been directly attached to the surgical drape material 116, the probe 120, or other component such as a wand, can be passed through the opening 118 in the surgical drape material 116, through the opening 106 formed in the proximal end 104 of the tubular sheath 102 of the probe cover 100, and into the channel 136 of the tubular sheath 102 until the probe 120 is located at the distal end 110 of the tubular sheath 102, where the distal end 110 is present in the sterile surgical field 112 for use by a surgeon or other healthcare professional.

A method for using the probe 120 in a sterile surgical field 112 via a probe cover 100 is discussed in more detail below with reference to FIG. 4. First, the probe cover 100 is positioned on the sterile surgical field 112 side of surgical drape material 116, then a non-sterile field facing surface 132 of the flange 108 is attached to the sterile surgical field facing surface 150 of the surgical drape material 116 via an adhesive 134 present on the non-sterile field facing surface 132 of the flange 108 or other suitable attachment means. In some embodiments, a release liner 138 (see FIGS. 1 and 3) must be removed so that the flange 108 can be attached via the adhesive 134. Next, the probe 120 is inserted into the opening 106 at the proximal end 104 of the tubular sheath 102 and into the channel 136 from a non-sterile field 114 side of the surgical drape material, wherein the probe is connected to the non-sterile medical instrument 124 via a cord 122 such that the probe 120 is encased by the tubular sheath 102.

In some embodiments, prior to or after attaching the non-sterile field facing surface 132 of the flange 108 to the sterile surgical field facing surface 150 of the surgical drape material 116, an opening 118 may need to be cut in the surgical drape material 116 at a location where the flange 108 is to be attached such that the opening 106 at the proximal end 104 of the tubular sheath 102 lines up with the opening 118 cut in the surgical drape material 116. In other embodiments, the opening 118 may be pre-formed in the surgical drape material 116 so that a cut does not need to be made. After the flange 108 is attached to the surgical drape material 116, the probe 120 is advanced into the sterile surgical field 112 for use on a patient. It is to be understood that the tubular sheath 102 and flange 108 are flexible such that the tubular sheath 102 is movable in multiple planes within the sterile surgical field 112 and such that the flange 108 is attachable to a planar or semi-planar surface, such as the sterile field facing surface 150 of the surgical drape material 116.

A method for using the probe 120 in a sterile surgical field 112 via a probe cover 100 is discussed in more detail below with reference to FIG. 5. First, the probe cover 100 is positioned on the non-sterile field 114 side of surgical drape material 116, then a sterile surgical field facing surface 130 of the flange 108 is attached to the non-sterile field facing surface 152 of the surgical drape material 116 via an adhesive 134 present on the sterile surgical field facing surface 130 of the flange 108 or other suitable attachment means. In some embodiments, a release liner 138 (see FIGS. 1 and 3) must be removed so that the flange 108 can be attached via the adhesive 134. Next, the probe 120 is inserted into the opening 106 at the proximal end 104 of the tubular sheath 102 and into the channel 136 from a non-sterile field 114 side of the surgical drape material, wherein the probe is connected to the non-sterile medical instrument 124 via a cord 122 such that the probe 120 is encased by the tubular sheath 102.

In some embodiments, prior to or after attaching the sterile surgical field facing surface 130 of the flange 108 to the non-sterile surgical field facing surface 152 of the surgical drape material 116, an opening 118 may need to be cut in the surgical drape material 116 at a location where the flange 108 is to be attached such that the opening 106 at the proximal end 104 of the tubular sheath 102 lines up with the opening 118 cut in the surgical drape material 116. In other embodiments, the opening 118 may be pre-formed in the surgical drape material 116 so that a cut does not need to be made. After the flange 108 is attached to the surgical drape material 116, the probe 120 is advanced into the sterile surgical field 112 for use on a patient. It is to be understood that the tubular sheath 102 and flange 108 are flexible such that the tubular sheath 102 is movable in multiple planes within the sterile surgical field 112 and such that the flange 108 is attachable to a planar or semi-planar surface, such as the sterile field facing surface 150 of the surgical drape material 116.

The present invention may be better understood with reference to the following examples.

### Example 1

One particular example of attaching the probe cover 100 to the surgical drape material 116 is accomplished by utilizing a probe cover 100 that includes a tubular sheath 102 made of polyurethane or isoprene that contains similar acoustic transparent properties. The tubular sheath 102 is formed to create a closed distal end 110 and an open proximal end 104 that is continuously formed into a square flange 108 having a length of 127 millimeters and a width of 127 millimeters or a circular flange 108 having a diameter of 127 millimeters. The probe cover 100 of Example 1 is attached to the sterile surgical field facing surface 150 of the surgical drape material 116 at its flange 108's non-sterile field facing surface 132 over the opening 118 in the surgical drape material 116. An adhesive 134 disposed on the non-sterile field facing surface 132 of the flange 108 is used to bond or adhere the flange 108 to the surgical drape material 116. A probe 120 or other medical instrument can then be inserted through the opening 118 in the surgical drape material 116, through the opening 106 at the proximal end 104 of the tubular sheath 102, and into the channel 136 that extends into the sterile surgical field 112 so that the probe 120 can be safely used in the sterile surgical field 112 so that the probe 120 can be used during a medical procedure.

### Example 2

Another particular example of attaching the probe cover 100 to the surgical drape material 116 is accomplished by utilizing a probe cover 100 that includes a tubular sheath 102 made of polyurethane or isoprene that contains similar acoustic transparent properties. As in Example 1, the tubular sheath 102 is formed to create a closed distal end 110 and an open proximal end 104 that is continuously formed into a square flange 108 having a length of 127 millimeters and a width of 127 millimeters or a circular flange 108 having a diameter of 127 millimeters. Unlike Example 1, however, the probe cover 100 of Example 2 is attached to the non-sterile field facing surface 152 of the surgical drape material 116 at its flange 108's sterile surgical field facing surface 130 over the opening 118 in the surgical drape material 116. An adhesive 134 disposed on the sterile surgical field facing surface 130 of the flange 108 is used to bond or adhere the flange 108 to the surgical drape material 116. Further, once attached, the tubular sheath 102 extend through the opening 118 in the surgical drape material 116 into the sterile surgical field 112, where a probe 120 or other medical instrument can be inserted through the opening 106 at the proximal end 104 of the tubular sheath 102, through the opening 118 in the surgical drape material 116, and into the channel 136 that extends into the sterile surgical field 112 so that the probe 120 can be used during a medical procedure.

The present invention has been described both in general and in detail by way of examples. These and other modifications and variations of the present invention may be practiced by those of ordinary skill in the art, without departing from the spirit and scope of the present invention. In addition, it should be understood that aspects of the various embodiments may be interchanged both in whole or in part. Furthermore, those of ordinary skill in the art will appreciate that the foregoing description is by way of example only, and is not intended to limit the invention so further described in such appended claims.

1. A probe cover formed from a film, wherein the film defines: (a) a tubular sheath having a distal end and a proximal end defining a channel therebetween, wherein the distal end is closed and the proximal end defines an opening having a periphery; and (b) a flange extending from the periphery of the opening at the proximal end of the tubular sheath.

2. The probe cover of claim 1, wherein the flange is adapted to attach to a planar or semi-planar surface.

3. The probe cover of claim 1, wherein the tubular sheath and the flange are flexible.

4. The probe cover claim 1, wherein an adhesive is disposed on a non-sterile field facing surface of the flange or a sterile surgical field facing surface of the flange.

5. The probe cover claim 4, wherein a release liner is disposed on the adhesive.

6. The probe cover of claim 4, wherein the adhesive comprises a hot melt adhesive coating, an acrylic adhesive coating, a tape, or a combination thereof.

7. The probe cover claim 1, wherein the probe cover is configured for direct attachment to a surgical drape.

8. The probe cover claim 1, wherein the film is seamless.

9. The probe cover claim 1, wherein the film comprises latex, polyurethane, polyisoprene, polycarbonate, polypropylene, polyethylene, or a combination thereof.

10. The probe cover claim 1, wherein the flange extends from the periphery of the opening at the proximal end of the tubular sheath to opposing first and second edges and opposing third and fourth edges such that the flange has a square or rectangular shape.

11. The probe cover claim 1, wherein the flange has a surface area that is sufficiently large to cover an opening in a surgical drape to which the flange is attached.

12. The probe cover claim 1, wherein the channel receives a probe connected to a non-sterile medical instrument.

13. The probe cover of claim 12, wherein the probe is an ultrasound probe, an echocardiography probe, or an internal cardiac defibrillator wand.

14. A method for using a probe in a sterile surgical field, wherein the probe is a component of a non-sterile medical instrument located in a non-sterile field, the method comprising:
a) positioning a probe cover adjacent a sterile surgical field side or a non-sterile field side of a surgical drape material, wherein the probe cover is formed from a film, wherein the film defines: (i) a tubular sheath having a distal end and a proximal end defining a channel therebetween, wherein the distal end is closed and the proximal end defines an opening; and (ii) a flange extending from the periphery of the opening at the proximal end of the tubular sheath;
b) attaching a non-sterile field facing surface of the flange to a sterile surgical field facing surface of the surgical drape material or attaching a sterile field facing surface of the flange to a non-sterile field facing surface of the surgical drape material; and
c) inserting the probe into the opening at the proximal end of the tubular sheath and into the channel from the non-sterile field, wherein the probe is connected to the non-sterile medical instrument via a cord such that the probe is encased by the tubular sheath.

15. The method of claim 14, wherein an adhesive is disposed on the non-sterile field facing surface of the flange or on the sterile surgical field facing surface of the flange.

16. The method of claim 15, wherein a release liner is disposed on the adhesive, wherein the release liner is removed prior to attaching the non-sterile field facing surface of the flange to the sterile surgical field facing surface of the surgical drape material or prior to attaching the sterile-field facing surface of the flange to the non-sterile field facing surface of the surgical drape material.

17. The method of claim 14, further comprising cutting an opening in the surgical drape material at a location where the flange is attached such that the opening at the proximal end of the tubular sheath lines up with the opening cut in the surgical drape material prior to or after step b).

18. The method of claim 17, wherein the flange has a surface area that is sufficiently large to cover the opening in the surgical drape material.

19. The method of any one of claim 14, further comprising d) advancing the probe contained within the channel and encased by the tubular sheath into the sterile surgical field for use on a patient.

20. The method of any one of claim 14, wherein the film is seamless.

21. The method of any one of claim 14, wherein the tubular sheath and flange are flexible such that the tubular sheath is movable in multiple planes within the sterile surgical field and such that the flange is attachable to a planar or semi-planar surface.

22. The method of any one of claim 14, wherein the probe is an ultrasound probe, an echocardiography probe, or an internal cardiac defibrillator wand.

23. A surgical drape for separating a sterile surgical field from a non-sterile field, the surgical drape comprising:
a surgical drape material having an opening formed therein; and
a probe cover formed from a film, wherein the film defines: (a) a tubular sheath having a distal end and a proximal end defining a channel therebetween, wherein the distal end is closed and the proximal end defines an opening having a periphery; and (b) a flange extending from the periphery of the opening at the proximal end of the tubular sheath, wherein the probe cover opening is aligned with the surgical drape material opening, wherein the probe cover is attached to the surgical drape material via the flange.
